(19) United States
(12) Patent Application Publication
(10) Pub. No.: US 2015/0089870 A1
(43) Pub. Date: Apr. 2, 2015

(54) POPULATION CONTROL OF INVASIVE PLANT SPECIES AND RESTORATION OF NATIVE PLANT COMMUNITIES

(71) Applicant: Wayne State University, Detroit, MI (US)

(72) Inventors: Edward M. GOLENBERG, Farmington Hills, MI (US); D. Carl FREEMAN, Troy, MI (US)

(21) Appl. No.: 14/503,675

(22) Filed: Oct. 1, 2014

Related U.S. Application Data
(60) Provisional application No. 61/885,095, filed on Oct. 1, 2013.

(51) Int. Cl. A01N 57/16; A01G 1/00; C12N 15/82

(52) U.S. Cl. CPC A01N 57/16; C12N 15/8218; A01G 1/001
USPC 47/58.1 R; 504/196

(57) ABSTRACT

The invention provides methods for controlling invasive plants, comprising treating one or more populations of invasive plant species with a composition comprising an effective amount of nucleic acids, wherein the nucleic acids have been produced using gene silencing techniques to target specific plant species, and specific aspects of plant development. In the methods of the invention, the compositions applied to the plants adversely affect only the targeted invasive plants, as compared to any non-invasive plants living in the same environmental setting. Further provided are methods for restoring native plant flora to an environment, by treating a natural environment using the methods provided herein to reduce or eliminate a species of invasive plant in an environment, then reintroducing a native plant species to the environment, such that the native plant population is reestablished in an environment and the invasive species is eliminated or reduced.

### CROSS-REFERENCE TO RELATED APPLICATION

This application claims benefit of US Provisional Application No. 61/885,095, filed on 1 Oct. 2013 and which application is incorporated herein by reference. A claim of priority is made.

### GOVERNMENT SUPPORT

This invention was made with government support under Grant/Contract No. G10AC00336 awarded by the U.S. Geological Survey, U.S. Department of the Interior. The United States Government may have certain rights in the invention.

### BACKGROUND OF THE INVENTION

Invasive species cost the United States billions of dollars annually and disrupt natural ecosystems. Across the U.S., invasive plants are estimated to occur on 7 million acres of national park lands, and at least 1.5 million acres are severely infested. In addition to federal lands, state and private lands are also plagued with invasive plants and may have even higher infestation rates.

Invasive plants can cause wildfires to occur more often and burn more intensely as a significant and potentially dry fuel source. The economic impact of invasive plants is estimated at more than $34 billion per year, and the costs continue to grow (http://www.mipn.org/InvasivesBrochure.pdf). Thus, invasive plants cause major negative impacts to our ecosystem and economy.

Invasive species threaten not only native species but also the benefits humans obtain from ecosystems. Phragmites (frag-MY-teez), a tall wetland grass, has been a component of wetlands in the U.S. for several thousand years. However, a novel genotype of Phragmites introduced from Europe began displacing the native genotype in early 20th century and rapidly spread across the continent. This genotype of Phragmites has become an invasive species in the U.S. The non-native Phragmites alters the soil, produces copious seeds (that can remain dormant for several years), and can propagate vegetatively, producing dense, nearly monoculture stands. As

Phragmites invades an area, plant species diversity declines and critical habitat for fish, reptiles, amphibians, and birds is lost. Presently, Phragmites has colonized many marshes throughout the eastern United States, is rapidly invading the few remaining marshes in the Great Lakes, and has spread to the Pacific coast, where it is again displacing local vegetation.

Conventional wisdom holds that introduced plants are largely unaffected by predators, pathogens, and other factors that would naturally regulate their density (i.e., “the enemies release” hypothesis). Native plants, having density-dependent regulation, are at a consequent competitive disadvantage to the non-native plants. Hence, invasive plants are thought to displace native plants simply because the introduced plants have fewer enemies.

Phragmites and other highly invasive plants continue to spread across the landscape and reduce the quality of habitat for wetland plants and animals, decrease the aesthetic value of property, and increase the likelihood of damaging fires. Current management approaches (e.g., spraying, burning, cutting) are resource-intensive, difficult to maintain over the long term and can have detrimental impacts on the surrounding environment. Because the current control strategies are time, labor, and resource intensive, innovative methods to control the spread of Phragmites or minimize its invasive properties are needed. In addition, sustainable strategies for landowners and resource managers are needed in order to control the population of invasive plant species and restore native plant communities.

### SUMMARY OF THE INVENTION

Applicants have discovered unique and effective methods for controlling or eradicating populations of invasive plants. In certain embodiments, methods for controlling a population of an invasive plant are provided, and the methods comprise treating a population of an invasive plant species in an environmental setting with a composition comprising an effective amount of polynucleotides, and said polynucleotides having been modified with a gene-silencing vector, and wherein the composition adversely affects the invasive plant species, as compared to non-invasive plant species living in the same environmental setting.

Also, the methods provided optionally comprise a cocktail containing one or more modified polynucleotides, wherein one or more invasive plant species are adversely affected, as compared to non-invasive plant species living in the same environment.

In certain embodiments of the methods provided herein, the non-invasive plant species are not adversely affected by the composition. In other embodiments, the polynucleotides are degradable via natural processes of degradation. Also, in certain methods provided herein, the polynucleotides comprise artificial microRNA (amiR) and in other methods, the polynucleotides are species-specific.

In some embodiments, the polynucleotides silence the genes involved in growth, seed production, vegetative reproduction, photosynthesis, flower organ development, pollen production, or a combination thereof, in the invasive plant species. In other embodiments of the methods provided herein, the polynucleotides comprise gene silencing vectors which inhibit or reduce the expression of the genes involved in growth, seed production , vegetative reproduction, photosynthesis, flower organ development, pollen production, or a combination thereof, in the invasive plant species.

In select methods, the population of the invasive plant species is eradicated in the environmental area treated. In other methods, the adverse effect is the loss of the invasive plant species' competitive advantage over the non-invasive plant species in the environment were the treatment occurred. In some embodiments of the methods of the invention, the invasive species is Phragmites.

Also provided are methods of inhibiting the expression of a target gene in an invasive plant species, comprising treating a population of an invasive plant species in an environmental setting with an effective amount of a composition comprising polynucleotides, wherein the polynucleotides have been modified with a vector that silences the target gene, and wherein the polynucleotides inhibit the expression of said target gene in said invasive plant species, wherein expression of said target gene is essential to growth, seed production, vegetative reproduction, photosynthesis, flower organ development, pollen production, or a combination thereof, of said invasive plant species, and wherein the composition does not affect non-invasive plant species in the same environmental setting.

Moreover, provided herein are methods for restoring native plant flora to an environment, comprising, treating a natural environment using the methods disclosed herein to reduce or eliminate an invasive plant species in a natural environment, and reintroducing a native plant species to the environment, so that native plant species is reestablished in the environment and the invasive plant species in the same environment is eliminated or reduced. In one embodiment of the methods disclosed herein, native plant species are not adversely affected by the composition. In addition, according to certain methods, dead zones are not created by the reduction of elimination of the invasive plant species in the natural environment, and in other methods, the invasive plant species loses its competitive advantage and is replaced gradually by the reintroduced native plant species.

Further, provided are compositions for controlling the population of an invasive plant species, comprising an effective amount of polynucleotides, said polynucleotides having been modified with a gene-silencing vector, and an agriculturally or a horticulturally acceptable carrier. According to certain embodiments, the compositions may also comprise a second effective amount of polynucleotides, said polynucleotides having been modified with a gene-silencing vector that is different from the first gene-silencing vector. In other embodiments, the compositions of the invention may control the population of the invasive plant species Phragmites.

Still other embodiments of the compositions provide for the polynucleotides to be comprised of artificial microRNA (amiR). In certain compositions, the gene-silencing vector silences one or more target genes. Additionally, the gene-silencing vectors silence target genes that are selected from the genes involved in growth, seed production, vegetative reproduction, photosynthesis, flower organ development, pollen production, or a combination thereof, in the invasive plant species. The compositions as provided herein can be in a liquid, liquid suspension, powder, granule, gel, or emulsion form, and optionally may contain comprises RNA stabilizing agents, RNA stabilizing carriers, or a combination thereof.

In another embodiment of the invention, methods are provided for controlling, reducing or eliminating a population of an invasive plant species that comprise treating a population of a species of invasive plant with a composition comprising an effective amount of nucleic acids, where the composition adversely affects the invasive plant, as compared to non-invasive plants living in the same environmental setting. In certain other embodiments, the methods of the invention provide treatment or control of more than one population of invasive plant. Further, in other embodiments of the methods of the invention, the composition comprises a mixture of nucleic acids, wherein so that one or more invasive plant species are adversely affected, as compared to non-invasive plants living in the same environment.

In still other embodiments of the methods of the invention, non-invasive species are not adversely affected by the composition used in the methods of the invention to control or eradicate invasive species. Also, in certain embodiments of the methods of the invention, the nucleic acids comprise artificial microRNA (amiR). In an another embodiment of the methods of the invention, the nucleic acids silence the genes involved in growth, seed production, vegetative reproduction, photosynthesis, flower organ formation or development, number of organs developed, pollen production, or a combination thereof, in the plant. In another embodiment of the methods of the invention, the nucleic acids interfere with the expression of the genes involved in growth, seed production, vegetative reproduction, or a combination thereof, in the plant or plants to be controlled or eradicated. In still other embodiments of the methods of the invention, the nucleic acids are species-specific.

In additional embodiments of the methods of the invention, the population of the invasive plant is eradicated. Further, in certain embodiments of the methods of the invention, the invasive species is Phragmites.

In one embodiment of the methods of the invention, the nucleic acids inhibit the ability of the invasive plant to produce seeds. Additionally, in an embodiment of the methods of the invention, the nucleic acids comprise gene silencing vectors which interfere with the expression of the genes involved in growth, seed production, vegetative reproduction, functioning , or a combination thereof, in the plant.

### DETAILED DESCRIPTION OF THE INVENTION

### Definitions

As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described.

The singular forms “a,” “an,” and “the” include plural reference unless the context clearly dictates otherwise. Thus, for example, a reference to “a plant” includes a plurality of such plants. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation.

The term “and/or” means any one of the items, any combination of the items, or all of the items with which this term is associated. The phrase “one or more” is readily understood by one of skill in the art, particularly when read in context of its usage.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by the skilled artisan, all numbers, including those expressing quantities of reagents or ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

The term “contacting” refers to the act of touching, making contact, or of bringing to immediate or close proximity, including at the cellular or molecular level, for example, to bring about a physiological reaction, a chemical reaction, or a physical change, in vitro, or in vivo. An “effective amount” refers to an amount effective to bring about a recited effect.

The phrases “genetic information” and “genetic material”, as used herein, refer to materials found in the nucleus, mitochondria and/or cytoplasm of a cell, which play a fundamental role in determining the structure and nature of cell substances, and capable of self-propagating and variation. The phrase “genetic material” of the present invention may be a gene, a part of a gene, a group of genes, DNA, RNA, nucleic acid, a nucleic acid fragment, a nucleotide sequence, a polynucleotide, a DNA sequence, a group of DNA molecules, double-stranded RNA (dsRNA), small interfering RNA or small inhibitory RNA (siRNA), or microRNA (miRNA)or the entire genome of an organism. The genetic material of the present invention may be naturally occurring.

As used herein, the term “nucleic acid” and “polynucleotide” refers deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res., 19:508 (1991); Ohtsuka et al., J. Biol. Chem., 260:2605 (1985); Rossolini et al., Mol. Cell. Probes, 8:91 (1994).

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic

DNA, and even synthetic DNA sequences. The term also includes sequences that include any of the known base analogs of DNA and RNA.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res., 19:508 (1991); Ohtsuka et al., JBC, 260:2605 (1985); Rossolini et al., Mol. Cell. Probes, 8:91 (1994)).

Deoxyribonucleic acid (DNA) in the majority of organisms is the genetic material while ribonucleic acid (RNA) is involved in the transfer of information contained within

DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. A “nucleic acid fragment” is a fraction of a given nucleic acid molecule. The terms “nucleic acid”, “nucleic acid molecule”, “nucleic acid fragment”, “nucleic acid sequence or segment”, or “polynucleotide” may also be used interchangeably with gene, cDNA, DNA and RNA encoded by a gene.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. “Naturally occurring” is used to describe an object that can be found in nature as distinct from being artificially produced. For example, nucleotide sequence present in an organism (including a virus), which can be isolated from a source in nature and which has not been intentionally modified by man in the laboratory, is naturally occurring.

The invention encompasses isolated or substantially purified nucleic acid compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

The term “chimeric” refers to any gene or DNA that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and is stably maintained. Transgenes may include, for example, DNA that is either heterologous or homologous to the DNA of a particular cell to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, N.Y.: Cold Spring Harbor Laboratory Press (3.sup.rd edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified.

The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Wild-type” refers to the normal gene, or organism found in nature without any known mutation.

“Genome” refers to the complete genetic material of an organism.

A “vector” is defined to include, inter alia, any plasmid, cosmid, phage or binary vector in double or single stranded linear or circular form which may or may not be self-transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides ('codon') in a coding sequence that specifies initiation and chain termination, respectively, of protein synthesis (mRNA translation).

A “functional RNA” refers to an antisense RNA, ribozyme, or other RNA that is not translated.

The term “RNA transcript” refers to the product resulting from RNA polymerase catalyzed transcription of a DNA sequence. When the RNA transcript is a perfect complimentary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., Mol. Biotech., 3:225 (1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

Thus, the genes and nucleotide sequences used in the methods of the invention include naturally occurring sequences as well as mutant, mutated or intentionally altered forms.

The terms “transfection” and “transformation”, as used herein, refer to the introduction of foreign DNA into eukaryotic or prokaryotic cells, or the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism into which a heterologous nucleic acid molecule has been introduced. The nucleic acid molecule can be stably integrated into the genome generally known in the art and are disclosed in Sambrook et al., Molecular Cloning: A Laboratory Manual (2d ed., Cold Spring Harbor Laboratory Press, Plainview, N.Y.) (1989). See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome.

The term “untransformed” refers to normal cells that have not been through the transformation process.

The genetic material used in the methods of the present invention may be of eukaryotic (including plant kingdom members), prokaryotic, fungal, archaeal or viral origin.

As used herein, “gene silencing” is a process whereby the genetic message necessary to code for particular proteins is blocked (silenced), thus blocking the development of specific traits. As a non-limiting example of gene silencing, if the genetic code essential for photosynthesis is targeted and silenced, a plant will not be able to harness the sun's energy and growth will be stunted.

As used herein, “controlling” a population of a plant species refers to the regulation and management of a plant species.

As used herein, “treating” a population of plants refers to, for example, the application or administration of a composition or compositions on or to a plant population or an environment where plants are found.

As used herein “adversely effects” is defined as a negative or detrimental effect on a plant population. When a plant population is adversely effected, the plant population may be eradicated, or it may be inhibited, for example, from growing, producing seed, vegetatively reproducing. Additionally, adverse effects on a plant population may include any detrimental effect on the growth, life, fitness, or survival of the plant population.

Applicants' invention utilizes molecular technology and plant developmental genetics for applications in ecology problems. Invasive plant species have both economic and ecological impacts on a national and an international scale. Invasive species replace native species due to competitive advantages. These advantages stem from a combination of lack of enemies (predators and pathogens), rapid growth of biomass that disproportionately controls limiting resources, high fecundity rates that allow for high seed production and establishment of new individuals, extensive vegetative reproduction that both controls physical space and permits colonization of new areas with few individuals, and production of secondary effects that degrade an environment for native species.

Phragmites australis, also known as common reed, is a perennial, wetland grass that can grow to 15 feet in height. It is a rhizoid plant with 80% of its mass underground. Non-native varieties of phragmites are becoming widespread and are threatening the ecological health of wetlands and the Great Lakes coastal shoreline. Invasive phragmites creates tall, dense stands which degrade wetlands and coastal areas by crowding out native plants and animals, blocking shoreline views, reducing access for swimming, fishing, and hunting and can create fire hazards from dry plant material.

The current means for control of invasive species are inadequate and ineffective. The current methods include costly, labor intensive efforts such as flooding, cutting, burning, and covering (using landscape fabric and the like), as well as applying herbicides.

As an example of the impact of one invasive species, a recent study of the operation and effectiveness of current Phragmites control programs found that an estimated $4.6 million USD is spent on mechanical and chemical Phragmites control on over 200,000 acres annually, with no statistical relationship between resources invested and management success. (Martin and Blossey 2013).

Applicants have developed novel, effective techniques and methods which provide innovative approaches to control invasive species. Applicants' methods involve silencing the genes that code for traits that enhance invasion and the species-specific competitive abilities of invasive plant species, such as, but not limited to, Phragmites.

Applicants' biological control methods involve species-specific reduction of the invasive and competitive abilities of invasive plant species, including, but not limited to, plants such as Phragmites. Applicants have also developed a gene-silencing vector, which has been successfully applied to many dicot plant species. This vector, as used in Applicants' methods, acts to suppress the activity of specific genes by targeting the initial products of the gene for degradation. As long as the exact gene sequence is known, any gene may theoretically be chosen for use in Applicants' methods and processes.

In addition, Applicants have demonstrated that genes involved in photosynthesis, flower organ formation, and organ number can be silenced in spinach, tobacco, and tomato. Applicants also have demonstrated that the vector can be applied either through biolistic or Agrobacterium applications. One advantage of the use of the vector in Applicants' methods is that it cannot spread either systemically through the plant or be transferred incidentally to other individuals, and thus should be safe to apply in an open area.

As a non-limiting example, Phragmites genes involved in flower production, and thus seed and pollen production, and in rhizome development, have been targeted and studied. By silencing these genes, colonization of new areas is reduced, the spread of established colonies through aggressive vegetative expansion is minimized, and native plant species are allowed to compete and eventually replace Phragmites without causing ancillary ecological disturbances.

In one embodiment, the methods of the invention involve a combination of two processes. First, the genes involved in the growth (photosynthesis and hormone production), seed production (floral developmental genes), and/or vegetative reproduction (root development and meristem developmental genes) of an invasive are silenced. Silencing is accomplished according to standard protocols, and according to certain embodiments of the invention, silencing signals are designed to be species-specific and gene specific. Second, the invention provides management protocols which provide for the reintroduction of native species in treated areas in order to accelerate ecological succession.

The methods of the invention utilize known gene silencing processes to control the spread of invasion plant species and to restore native plant communities. Gene silencing is a technique that utilizes gene regulation machinery common among eukaryotic organisms. Molecules of double-stranded RNA, dsRNA, with specific conformational and energetic properties, trigger a pathway that initially cleaves the dsRNA into small, 21 or 22 nucleotide segments, and then the 21 nucleotide segments are used to interfere with the expression of target genes. The genes that are targeted for interference are based on the identity of the 21 nucleotide segments. Because the triggering silencing signals are so small, it is possible to design signals that are not only gene specific, but also species specific.

Applicants' methods can be utilized to combat any invasive plant species. A non-limiting list of invasive plant species to which Applicants invention may be applied to in the United States and North America are as follows: Achillea species, including A. millefolium (Common Yarrow); Acroptilon species, including A. repens (Russian knapweed); Ailanthus species, including A. altissima (chouchun, tree of heaven); Albizia species, including A. julibrissin (Persian silk tree, pink sins); Alliaria species, including A. petiolata (garlic mustard, hedge garlic); Alnus species, including A. glutinosa (Black Alder); Anthemis species, including A. cotula (Mayweed); Ardisia species, including A. crenata (coral bush, coralberry); Arundo species, including A.donax (giant reed, Spanish cane); Barbarea species, including B. verna (Land Cress); Barbarea species, including B. vulgaris (Yellow Rocket); Berberis species, including B. thunbergii (Japanese barberry, Thunberg's barberry); Bromus species, including B. tectorum (downy brome, drooping brome); Butomus species, including B. umbellatus (flowering rush, grass rush); Cardamine species, including C. hirsuta (Hairy Bittercress); Carduus species, including C. nutans (musk thistle, nodding thistle); Casuarina species, including C. equisetifolia (Australian pine, Filao tree); Caulerpa species, including C. taxifolia (“killer algae”); Celastrus species, including C. orbiculatus (Oriental bittersweet); Centaurea species, including C. diffusa (diffuse knapweed, white knapweed); Centaurea species, including C. maculosa (spotted knapweed); Centaurea species, including C. solstitialis (yellow cockspur, yellow starthistle); Chrysanthemum species, including C. leucanthemum (Oxeye Daisy); Cinnamomum species, including C. camphora (camphor laurel, camphor tree); Cirsium species, including C. arvense (Canada Thistle); Cirsium species, including C. vulgare (Bull Thistle); Colocasia species, including C. esculenta (wild taro); Commelina species, including C. communis (Asiatic blue dayflower); Conium species, including C. maculatum (poison hemlock); Cortaderia species, including C. selloana (pampas grass); Cosmos species, including C. sulphureus (sulfur cosmos, yellow Cosmos); Cynanchum species, including C. louisae; Cynanchum species, including C. rossicum (dog-strangling vine, swallowwort); Cytisus species, including C. scoparius (common broom, Scotch broom); Daucus species, including D. carota (Queen Anne's Lace); Dioscorea species, including D. bulbifera (air potato); Egeria species, including E. densa (Brazilian waterweed, large-flowered waterweed); Eichhornia species, including E. crassipes (common water hyacinth); Elaeagnus species, including E. angustifolia (Russian olive, Russian silverberry); Elaeagnus species, including E. umbellata (autumn olive, Japanese silverberry); Eucalyptus species, including E. globulus (southern blue gum, Tasmanian blue gum); Euonymus species, including E. alata (burning bush, winged euonymus); Euonymus species, including E. fortunei (winter creeper vine); Euphorbia species, including E. esula (green spurge, leafy spurge); Glechoma species, including G. hederacea (Ground Ivy); Fallopia species, including F. japonica (fleeceflower, Japanese knotweed); Hedera species, including H. helix (common ivy, English ivy); Heracleum species, including H. mantegazzianum (giant cow parsley, giant hogweed); Hesperis species, including H. matronalis (Dame's Rocket); Hieracium species, including H. aurantiacum (orange hawkweed, tawny hawkweed); Hydrilla species, including H. verticillata (Esthwaite waterweed, hydrilla); Imperata species, including I. cylindrica (cogon, cogongrass); Ligustrum species, including L. sinense (Chinese privet); Ligustrum species, including L. vulgare (European privet, wild privet); Lonicera species, including L. japonica (Japanese honeysuckle); Lonicera species, including L. maackii (Amur honeysuckle); Lonicera species, including L. morrowii (Morrow's honeysuckle); Lonicera species, including L. tatarica (Tartarian honeysuckle); Lygodium species, including L. japonicum (Japanese climbing fern); Lygodium species, including L. microphyllum (Old World climbing fern, climbing maidenhair); Lysimachia species, including L. nummularia (creeping Jenny, herb twopence, moneywort, twopenny grass); Lythrum species, including L. salicaria (purple loosestrife, purple lythrum, spiked loosestrife); Melaleuca species, including M. quinquenervia (broad-leaved paper bark, paper bark tea tree, niaouli); Melinis species, including M. repens (Natal grass, Natal redtop, rose Natal grass); Melia species, including M. azedarach (bead tree, ceylon cedar, Chinaberry, lunumidella, Persian lilac, white cedar); Microstegium species, including M. vimineum (Japanese stiltgrass, Nepalese browntop); Mimosa species, including M. pudica (humble plant, shameful plant, sensitive plant, sleeping grass, touch-me-not); Myriophyllum species, including M. spicatum (Eurasian water milfoil, Spiked water milfoil); Onopordum species, including O. acanthium (cotton thistle, heraldic thistle, Scots thistle, Scottish thistle, woolly thistle); Paederia species, including P. foetida (skunk vine); Panicum species, including P. repens (torpedo grass); Pastinaca species, including P. sativa (parsnip); Paulownia species, including P. tomentosa (princess tree); Persicaria species, including P. perfoliata * (formerly Polygonum perfoliatum; Asiatic tearthumb, Chinese tearthumb, devil shield, devil's tail tearthumb, mile-a-minute weed); Persicaria species, including P. vulgaris * (pink lady's thumb); Phragmites species, including P. australis (common reed)[1]; Plantago species, including P. major (Broadleaf Plantain); Potamogeton P. crispus (curly-leaf pondweed); Pueraria species, including P. lobata (kudzu); Ranunculus species, including R. ficaria (Lesser Celandine); Rosa species, including R. multiflora (baby rose, multiflora rose, rambler rose); Rubus species, including R. armeniacus (Armenian blackberry, Himalayan blackberry); Rubus species, including R. phoenicolasius (Japanese wineberry, wine raspberry, wineberry); Rumex species, including R. crispus (curled dock, curly dock, narrow dock, sour dock, yellow dock); Salvinia species, including S. molesta (giant salvinia, kariba weed); Schinus species, including S. terebinthifolius (aroeira, Brazilian pepper, Christmasberry, Florida holly, rose pepper); Solanum species, including S. viarum (tropical soda apple); Solanum species, including S. dulcamara (Woody Nightshade); Solanum species, including S. nigrum (Deadly Nightshade); Spartina species, including S. alterniflora (saltmarsh cordgrass, smooth cordgrass); Striga species, including S. asiatica (Asiatic witchweed); Tamarix species (saltcedar, tamarisk); Tanacetum species, including T. vulgare (Common Tansy); Trapa species, including T. natans (water caltrop, water chestnut); Triadica species, including T. sebifera (Sapium sebiferum; Chinese tallow tree, Florida aspen, Gray popcorn tree); Verbascum species, including V. thapsus (Common Mullein); Vinca species, including V. minor (Small Periwinkle). The before-listed species represent only an example of invasive plant species that may be used in the methods of Applicants' invention.

Applicants' invention is superior to other methods in that 1) it is designed to only affect target species; 2) it does not create dead zones; 3) it does not use herbicides or off-target chemicals (thus has no effect on non-target species; also, in certain application methods, it does not cause spray drift and will not affect animals or plants in adjoining area); and 4) it incorporates a management protocol which includes introduction of native plants to succeed the invasive plant so that a dead zone is not created. According to the methods provided herein, the targeted species loses its competitive advantage and is replaced in a gradual manner by native species, with no dead zone created by the loss of the targeted species. Also, because the invention can directly target the production of seeds, certain embodiments of the invention provide for the reduction in the seed bank of the invasive species, while the same species is being replaced.

The methods of the invention employ nucleic acids to treat plants. Nucleic acids are degradable naturally in the environment and are not likely to cause residual, long-term negative ecological effects.

The nucleic acids used in the methods of the invention can be in any form desired, provided the form does not interfere with the effectiveness of the methods. For example, the nucleic acids may be in the following forms: liquid, liquid suspension, powder, granule, gel, emulsion, and the like, with or without RNA stabilizing agents or carriers. The nucleic acids used in the methods of the invention may be stored in a concentrated form, and may be diluted prior to use or application. The nucleic acids used in the methods of the invention may be applied to plants, for example, via spray, dusting, painting, particle bombardment, and by any other means.

The compositions as used in the methods of the invention include nucleic acids and other materials that do not interfere with the effectiveness of the methods.

In another embodiment, Applicants' methods and technology can be applied in agricultural and horticultural settings to control weeds or manage artificial plant communities. In a further embodiment, Applicants' methods provide silencing cocktails for specific target species within specific environments. An advantage offered by Applicants' methods for silencing cocktails is the relatively low cost. In another embodiment, Applicants' methods can easily be scaled for large area applications by using common biotechnology techniques through the growth of bacteria in order to produce necessary quantities of needed plasmids or other nucleic acid materials, including double stranded RNA or viral vectors.

The methods and technologies of the invention are applicable to all terrestrial invasive plant species, and therefore are able to be used and scaled for a variety of environments.

The invention provides gene specific silencing vectors, which can be designed for any invasive plant species.

Applicants' methods exploit artificial microRNA (amiR) technologies to provide species specific combinations in order to, for example, silence growth, expansion, and/or seed production genes in plants. These methods provide for the reduction and eventual eradication of invasive plant species, including but not limited to Phragmites. In addition, Applicants' technology can be utilized to design vectors that would reduce and eradicate other invasive plant species.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1: Modes of Applications to Elicit Transient Expression

Transient expression in plant cells is achieved by introducing a plasmid with a strong promoter into the nucleus of the cell. Common techniques can be utilized to introduce gene silencing vectors into host plants. As non-limiting examples, two commonly used techniques for introducing gene silencing vectors into host plants are Agrobacterium tumifaciens transformation and biolistic bombardment. Both techniques have been utilized for over twenty years. Recent reviews that discuss their use for transient expression are by Saunders and Lomonossoff (Saunders and Lomonossoff, 2013) and Sudowe et al. (2013).

### Example 2: Genes for Targeted Silencing

To reduce competitive abilities of invasive plants, gene involved in seed production, growth and biomass production, and vegetative reproduction are targeted. As provided in the invention, the genes used in any specific method for any specific invasive species will vary, due to gene sequence suitability in a particular organism and the response achieved in trials. The following are non-limiting examples of approaches and genes can be selected for knock-downs based on known plant physiological and developmental processes. This list is provided for example purposes only, and therefore, is not an exhaustive or restrictive list.

To reduce seed production: Targeted genes for silencing are those required for gynoecium (ovary, pistil, and style) development and stamen production. Target genes include floral organ identity genes required for floral organ identity, including but not limited to AGAMOUS and SEPALATA homologues. Inactivation of these genes is known to either completely sterilize or block pollen production, respectively. This is well documented from classic work in Arabidopsis and Antirrhinum (Coen and Meyerowitz, 1991; Meyerowitz et al., 1991), as well as from Applicants' work in spinach (Sather et al., 2010). Additionally further upstream genes such as LEAFY can be silenced to suppress flower development (Wagner et al., 1999).

To reduce growth and biomass: Targeted genes for silencing are those involved in photosynthesis or vegetative growth. For example, multiple photosynthetic genes have been silenced as demonstrations of gene silencing effects and are therefore obvious targets. These include, but are not limited to, Phytoene desaturase (Burch-Smith et al., 2006; Gould and Kramer, 2007; Di Stilio et al., 2010), Mg chelatase(Chen et al., 2003), and rbcS (Golenberg et al., 2009). Further, plant growth can be reduced by silencing genes involved in cell expansion via regulation of phytohormones such as Giberillic Acid (GA) or by reducing lignin or cellulose production controlled by genes such as Brittle Culm (Aohara et al., 2009) and the like. Targeted genes for reducing or inhibiting root growth include genes involved in root organization (Sarkar et al., 2007; Deveaux et al., 2008; Stahl et al., 2009), as well genes involved with auxin transporters such as PINOID related genes (Okada et al., 1991; Vernoux et al., 2000) and the like.

### Example 3: Experimental Protocols

Currently, there are 359 nucleotide sequences listed in GenBank for Phragmites matrons. The majority of these sequences are chloroplast sequences used in phylogenetic analyses, microsatellite sequences, or highly conserved ribosomal protein sequences. There are no entries for floral or root developmental genes that may be used for species-specific RNAi knockdowns. To effect seed-set, we will focus on the floral organ identity genes involved in stamen and carpel formation, specifically the AGAMOUS homologue. To effect root growth, we will focus on homologues of SCARECROW, SHORT ROOT, and/or PLETHORA. To downregulate energy acquisition, we will focus on rbcS and MG chelatase

Degenerate primers can be designed based on published sequences from within the Poaceae. Specifically for AGAMOUS, the following sequences will be used: Zea ZAG1(NM_{—}001111851.1), ZAG2(NM_{—}001111908.1), Oryza Os01g0886200 (NP 001045028.1). For the SCARECROW homologue, we will use the following: Zea sc11 (541704),sc123 (542138), Oryza 0J1003C07.9, Ozsa7075. For SHORT ROOT, we will use Oryza 4343769, 4343753. For Mg Chelatase, we will use: Zea oyl (732841), Oryza 4332690, 4344148, 4332690. rbcS is available for multiple species and will be downloaded. Putative Phragmites homologues will be cloned initially into pGEM Teasy after amplification with an error reducing polymerase (eg. Phusion or Advantage Taq) and will be sequenced. Standard gene phylogenetic analyses will be used to confirm homology.

pWSRi vectors have been developed into forms that can be applied biolistically or via Agrobacterium (pWSRiA). The efficacy of these vectors will be tested in Phragmites using the genes cloned as described above. Phenotypic markers and mRNA levels will be tested. Experimental Design: The genes cloned as provided above will be subcloned into the pWSRi vectors. In Applicants' experience, fragments of approximately 200-250 by work well in knockdown experiments. Regions that are specifically highly variable among species to generate species-specific effects in Phragmites will be identified. Multiple regions will be used per gene if possible. The pWSRi constructs will be applied to plants via a gene gun. Alternatively, Agrobacterium strains will be transformed with the pWSRiA vectors and applied to seedling leaves using syringes without needles. Plants will initially be tested for the rbcS and chelatase genes, due to ease of phenotypic effect detection. Affected tissue will then be harvested to extract total RNA. Actual knockdown of the specific mRNAs will be determined using qRT-PCR. The Ambion 18S-competimers will be used as the internal control. Optionally, a housekeeping gene such as G6pdh can be used. Effects on root growth and flowering will be more time-consuming. The root growth will be determined in plants grown in agar or vermiculite. Root mass will be observed and then weighed when visible effects are detected. Flowering effects will be detected visually. Similar confirmation using qRT-PCR will be used.

### Example 4: Sample Application Regime and Effects

The role of gene silencing in invasive species control is to reduce or inhibit the competitive advantage of invasive species in a species-specific manner while not killing the plants. Reduced competitiveness allows native plant species to reestablish and supplant invasive plant species without creating disturbed dead zones. Management approaches require multiple applications of the compositions of the invention throughout the growing season to effect emergent traits in the target invasive plant populations. As a non-limiting example, early season growth of an invasive plant species could be reduced through applications targeting cell elongation, photosynthesis, etc. Reduction of seed production can be achieved by applications of gene specific knock-downs during the time of transition to flowering. The timing of such transitions varies among plant species due to their specific responses to environmental cues. Additionally, as seed banks present a source for re-recruitment of invasive species, multiple year management, using the methods and compositions provided herein, will be required.

### REFERENCES

1. Aohara, T., Kotake, T., Yasuko Kaneko, Takatsuji, H., Tsumuraya, Y., and Kawasaki, S. (2009). Rice BRITTLE CULM 5 (BRITTLE NODE) is Involved in Secondary Cell Wall Formation in the Sclerenchyma Tissue of Nodes. Plant Cell Physiol. 50, 1886-1897.

2. Burch-Smith, T. M., Schiff, M., Liu, Y., and Dinesh-Kumar, S. P. (2006). Efficient Virus-Induced Gene Silencing in Arabidopsis. Plant Physiol. 142, 21-27.

3. Chen, S., Hofius, D., Sonnewald, U., and Bornke, F. (2003). Temporal and spatial control of gene silencing in transgenic plants by inducible expression of double-stranded RNA. Plant J 36, 731-740.

4. Coen, E. S., and Meyerowitz, E. M. (1991). The war of the whorls: genetic interactions controlling flower development. Nature 353, 31-37.

5. Deveaux, Y., Toffano-Nioche, C., Claisse, G., Thareau, V., Morin, H., Laufs, P., Moreau, H., Kreis, M., and Lecharny, A. (2008). Genes of the most conserved WOX Glade in plants affect root and flower development in Arabidopsis. BMC Evol Biol 8, 291.

6. Di Stilio, V. S., Kumar, R. A., Oddone, A. M., Tolkin, T. R., Salles, P., and McCarty, K. (2010). Virus-induced gene silencing as a tool for comparative functional studies in Thalictrum. PLoS ONE 5, e12064.

7. Golenberg, E. M., Sather, D. N., Hancock, L. C., Buckley, K. J., Villafranco, N. M., and Bisaro, D. M. (2009). Development of a gene silencing DNA vector derived from a broad host range geminivirus. Plant Methods 5, 9.

8. Gould, B., and Kramer, E. (2007). Virus-induced gene silencing as a tool for functional analyses in the emerging model plant Aquilegia (columbine, Ranunculaceae). Plant Methods 3, 6.

9. Meyerowitz, E. M., Bowman, J. L., Brockman, L. L., Drews, G. N., Jack, T., Sieburth, L. E., and Weigel, D. (1991). A genetic and molecular model for flower development in Arabidopsis thaliana. Dev Suppl 1, 157-167.

10. Okada, K., Ueda, J., Komaki, M., Bell, C., and Shimura, Y. (1991). Requirement of the

Auxin Polar Transport System in Early Stages of Arabidopsis Floral Bud Formation. The Plant cell 3, 677-1361.

11. Ossowski, S., Schwab, R., and Weigel, D. (2008). Gene silencing in plants using artificial microRNAs and other small RNAs. The Plant Journal 53, 674-690.

12. Sarkar, A. K., Luijten, M., Miyashima, S., Lenhard, M., Hashimoto, T., Nakajima, K.,

Scheres, B., Heidstra, R., and Laux, T. (2007). Conserved factors regulate signalling in Arabidopsis thaliana shoot and root stem cell organizers. Nature 446, 811-814.

13. Sather, D. N., Jovanovic, M., and Golenberg, E. M. (2010). Functional analysis of B and C class floral organ genes in spinach demonstrates their role in sexual dimorphism. BMC Plant Biol 10, 46.

14. Saunders, K., and Lomonossoff, G. P. (2013). Exploiting plant virus-derived components to achieve in planta expression and for templates for synthetic biology applications. New Phytologist, n/a-n/a.

15. Schwab, R., Ossowski, S., Riester, M., Warthmann, N., and Weigel, D. (2006). Highly Specific Gene Silencing by Artificial MicroRNAs in Arabidopsis. Plant Cell 18, 1121-1133.

16. Stahl, Y., Wink, R. H., Ingram, G. C., and Simon, R. (2009). A Signaling Module Controlling the Stem Cell Niche in Arabidopsis Root Meristems. Current Biology 19, 909-914.

17. Sudowe, S., Reske-Kunz, A. B., Ueki, S., Magori, S., Lacroix, BJE., and Citovsky, V. (2013). Transient Gene Expression in Epidermal Cells of Plant Leaves by Biolistic DNA Delivery. In Biolistic DNA Delivery (Humana Press), pp. 17-26.

18. Vernoux, T., Kronenberger, J., Grandjean, O., Laufs, P., and Traas, J. (2000). PIN-FORMED 1 regulates cell fate at the periphery of the shoot apical meristem. Development 127, 5157-5165.

19. Wagner, D., Sablowski, R. W. M., and Meyerowitz, E. M. (1999). Transcriptional activation of APETALAl by LEAFY. Science 285, 582-584.

All publications, patents, and patent documents are incorporated by reference herein, as though individually incorporated by reference.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

What is claimed is:

1. A method for controlling a population of an invasive plant, comprising treating a population of an invasive plant species in an environmental setting with a composition comprising an effective amount of polynucleotides, said polynucleotides having been modified with a gene-silencing vector, and wherein the composition adversely affects the invasive plant species, as compared to non-invasive plant species living in the same environmental setting.

2. The method of claim 1, wherein composition comprise a cocktail containing one or more modified polynucleotides, wherein one or more invasive plant species are adversely affected, as compared to non-invasive plant species living in the same environment.

3. The method of claim 1, wherein non-invasive plant species are not adversely affected by the composition.

4. The method of claim 1, wherein the polynucleotides are degradable via natural processes of degradation.

5. The method of claim 1, wherein the polynucleotides comprise artificial microRNA (amiR).

6. The method of claim 1, wherein the polynucleotides silence the genes involved in growth, seed production, vegetative reproduction, photosynthesis, flower organ development, pollen production, or a combination thereof, in the invasive plant species.

7. The method of claim 1, wherein the polynucleotides comprise gene silencing vectors which inhibit or reduce the expression of the genes involved in growth, seed production , vegetative reproduction, photosynthesis, flower organ development, pollen production, or a combination thereof, in the invasive plant species.

8. The method of claim 1, wherein the polynucleotides are species-specific.

9. The method of claim 1, wherein the population of the invasive plant species is eradicated in the environmental area treated.

10. The method of claim 1, wherein the adverse effect is the loss of the invasive plant species' competitive advantage over the non-invasive plant species in the environment were the treatment occurred.

11. The method of claim 1, wherein the invasive species is Phragmites.

12. A method of inhibiting the expression of a target gene in an invasive plant species, comprising treating a population of an invasive plant species in an environmental setting with an effective amount of a composition comprising polynucleotides, wherein the polynucleotides have been modified with a vector that silences the target gene, and wherein the polynucleotides inhibit the expression of said target gene in said invasive plant species, wherein expression of said target gene is essential to growth, seed production, vegetative reproduction, photosynthesis, flower organ development, pollen production, or a combination thereof, of said invasive plant species, and wherein the composition does not affect non-invasive plant species in the same environmental setting.

13. A method for restoring native plant flora to an environment, comprising:
a. treating a natural environment using the method of claim 1 to reduce or eliminate an invasive plant species in a natural environment, and
b. reintroducing a native plant species to the environment, such that the native plant species is reestablished in the environment and the invasive plant species in the same environment is eliminated or reduced.

14. The method of claim 13, wherein native plant species are not adversely affected by the composition.

15. The method of claim 13, wherein dead zones are not created by the reduction of elimination of the invasive plant species in the natural environment.

16. The method of claim 13, wherein the invasive plant species loses its competitive advantage and is replaced gradually by the reintroduced native plant species.

17. A composition for controlling the population of an invasive plant species, comprising an effective amount of polynucleotides, said polynucleotides having been modified with a gene-silencing vector, and an agriculturally or a horticulturally acceptable carrier.

18. The composition of claim 17, further comprising a second effective amount of polynucleotides, said polynucleotides having been modified with a gene-silencing vector that is different from the gene-silencing vector of claim 17.

19. The composition of claim 17, wherein the invasive plant species is Phragmites.

20. The composition of claim 17, wherein the polynucleotides comprise artificial microRNA (amiR).

21. The composition of claim 17, wherein the gene-silencing vector silences one or more target genes.

22. The composition of claim 20, wherein the target genes are selected from the genes involved in growth, seed production, vegetative reproduction, photosynthesis, flower organ development, pollen production, or a combination thereof, in the invasive plant species.

23. The composition of claim 20, wherein the composition is in a liquid, liquid suspension, powder, granule, gel, or emulsion form.

24. The composition of claim 20, wherein the composition further comprises RNA stabilizing agents, RNA stabilizing carriers, or a combination thereof.
